(19) United States
(12) Patent Application Publication
(10) Pub. No.: US 2025/0090175 A1
(43) Pub. Date: Mar. 20, 2025

(54) EMBOLIZATION DEVICE

(71) Applicant: MEDSTAR HEALTH, INC., Columbia, MD (US)

(72) Inventor: Jeffrey C. Mai, Columbia, MD (US)

(21) Appl. No.: 18/291,947

(22) PCT Filed: Aug. 3, 2022
(86) PCT No.: PCT/US2022/039284

§ 371 Date: Jan. 25, 2024

Related U.S. Application Data
(60) Provisional application No. 63/228,676, filed on Aug. 3, 2021.

(51) Int. Cl. A61B 17/12; A61B 17/00

(52) U.S. Cl. CPC A61B 17/12113; A61B 17/12031; A61B 17/12145; A61B 2017/00867; A61B 2017/00876

(57) ABSTRACT

An embolization device including a filament or hypotube having a longitudinal axis extending therein and a plurality of notches distributed in a non-uniform. computationally derived pattern on the filament or hypotube. The pattern of notches at least partially contributes to the shape of the embolization device in a deployed state by selectively biasing the embolization device to assume a pre-defined shape. The pre-defined shape of the embolization device. in turn. is based on the configuration of the bodily cavity in which the embolization device is deployed.

### CROSS-REFERENCE TO RELATED APPLICATIONS

The present application claims priority to U.S. Provisional Application No. 63/228,676 filed on Aug. 3, 2021 and incorporated by reference herein.

### TECHNICAL FIELD

The present disclosure relates to an embolization device to obliterate an aneurysm.

### BACKGROUND

Endovascular interventional procedures for providing an artificial embolism are desirable in some patients for controlling internal bleeding, preventing blood supply to tumors or vascular malformations, or relieving pressure in the vessel wall associated with an aneurysm. Several approaches are known for facilitating an artificial embolism, including the use of an inflatable, detachable balloon or the injection of a coagulative substance. Another approach utilizes an occlusive wire coil and delivery system for positioning the coil at a desirable site in a blood vessel. Regarding the latter approach, an embolization coil is typically delivered to a desired location in the vasculature of a patient through use of a catheterization procedure. In this procedure, a catheter is first inserted into the vasculature of a patient and positioned to be proximal to the desired or targeted location. Then, a coil is loaded into the lumen of the catheter and advanced through the catheter using a “push” rod until it reaches and exits through the distal end of the catheter, whereupon the coil is detached. Techniques to deploy coils involve mechanical, electrical, chemical or hydraulic release systems. Upon deposition within the aneurysm or vessel, the coils initiate a thrombotic reaction that leads to luminal occlusion. If successful, this can prevent bleeding from the aneurysm or blockade of blood flow through the vessel. In the case of broad-based aneurysms, a stent may be passed first into the parent artery to serve as a scaffold for the coils.

### SUMMARY

In certain aspects, the present disclosure relates to an embolization device to occlude a cavity within the body, such as an aneurysm or vessel lumen. The embolization device can include a filament or hypotube having a longitudinal axis extending therein. A plurality of notches can be distributed in a non-uniform pattern along the filament or hypotube. This pattern at least partially contributes to the shape of the embolization device in a deployed state by selectively biasing the embolization device to assume a pre-defined conformation. The pre-defined shape is based on the configuration of the cavity in which the embolization device is deployed. The precise pattern of the notches can be computationally derived to achieve a pre-defined shape of the embolization device when subject to axial load or deposited within a constrained space. The non-uniform pattern of the notches can include non-uniform spacing of the notches as well as a non-uniform pattern of depths, shapes, orientations, and/or angles of the plurality of notches. Such non-uniform patterns can influence the constrained curvature of the embolization device when deployed. For example, the orientation along a given surface, width, depth, and/or periodicity of the notches can deliberately bias or skew the curvature of the embolization device under a loading force. As such, embolization devices as disclosed herein allow for precise control over the configuration of the device when deployed in a constrained space, such as an aneurysm or lumen, which can be advantageous in sufficiently occluding volumes of differing diameters and shapes.

### BRIEF DESCRIPTION OF THE DRAWINGS

FIG. 1 is a side view of a hypothetical device having a uniform pattern of notches along at least a part of the length thereof.

FIG. 2 is a side view of an embolization device having a non-uniform pattern of notches along at least a part of the length thereof.

FIG. 3A is perspective view an exemplary embolization device (or part thereof) that has a pre-defined shape according to an aspect of the present disclosure.

FIG. 3B is a side view of the embolization device of FIG. 3A where the embolization device is in a substantially linear configuration such as when it is loaded into a delivery device.

FIG. 3C is perspective view of the embolization device of FIG. 3A when the embolization device is deployed within an aneurysm.

FIG. 4A is a side view of an embolization device (or part thereof) when the embolization device is in a substantially linear configuration such as when it is loaded into a delivery device according to an aspect of the present disclosure.

FIG. 4B is a perspective view of a part of the embolization device of FIG. 4A in a deployed state.

FIG. 5A is a side view of an embolization device (or part thereof) when the embolization device is in a substantially linear configuration such as when it is loaded into a delivery device according to an aspect of the present disclosure.

FIG. 5B is a side view of the embolization device of FIG. 5A in a deployed state or intermediate semi-deployed state.

FIG. 6A is a side view of an embolization device (or part thereof) when the embolization device is in a substantially linear configuration such as when it is loaded into a delivery device according to an aspect of the present disclosure.

FIG. 6B is a side view of the embolization device of FIG. 6A in a deployed state or intermediate semi-deployed state.

FIG. 7 is a perspective view of an embolization device when deployed into an aneurysm according to an aspect of the present disclosure.

FIG. 8 is a side view of a part of a filament or hypotube according to an aspect of the present disclosure.

FIG. 9 is a side view of an embolization device (or part thereof) in a substantially linear configuration such as when it is loaded into a delivery device according to an aspect of the present disclosure.

FIG. 10 is a perspective view of an embolization device (or part thereof) in a substantially linear configuration such as when it is loaded into a delivery device according to an aspect of the present disclosure.

FIG. 11 is a perspective view of an embolization device (or part thereof) in a substantially linear configuration such as when it is loaded into a delivery device according to an aspect of the present disclosure.

FIG. 12A is side view of an embolization device (or part thereof) in a substantially linear configuration such as when it is loaded into a delivery device according to an aspect of the present disclosure.

FIG. 12B is a perspective view of the embolization device of FIG. 12A in a deployed or intermediate semi-deployed state.

FIG. 13 is a partial perspective view of an embolization device according to an aspect of the present disclosure.

FIG. 14A-14C illustrate an exemplary method of manufacturing an embolization device according to an aspect of the present disclosure.

FIG. 15 is a perspective view of an embolization device according to an aspect of the present disclosure.

FIG. 16 is a perspective view of an embolization device according to an aspect of the present disclosure.

FIG. 17 is a side view of an an embolization device according to an aspect of the present disclosure.

FIG. 18 is a perspective view of an embolization device according to an aspect of the present disclosure.

FIG. 19 is a side view of an embolization device according to an aspect of the present disclosure.

FIG. 20 is a side view of the embolization device of FIG. 19.

FIG. 21 is a perspective view of an embolization device according to an aspect of the present disclosure.

FIG. 22 is a side view of an embolization device according to an aspect of the present disclosure.

FIG. 23 is an opposing side view of the embolization device of FIG. 22.

FIG. 24 is a top view of the embolization device of FIG. 22.

### DETAILED DESCRIPTION

As used herein with respect to a described element, the terms “a,” “an,” and “the” include at least one or more of the described element including combinations thereof unless otherwise indicated. Further, the terms “or” and “and” refer to “and/or” and combinations thereof unless otherwise indicated. By “substantially” is meant that the distance, shape, or configuration of the described element need not have the mathematically exact described distance, shape, or configuration of the described element but can have a distance, shape, or configuration that is recognizable by one skilled in the art as generally or approximately having the described distance, shape, or configuration of the described element. The terms “first,” “second,” etc. are only used to distinguish one element from another unless indicated otherwise. Thus, a “first” element described below could also be termed a “second” element. By “integral” or “integrated” is meant that the described components are fabricated as one piece or multiple pieces affixed during manufacturing or the described components are otherwise not separable using a normal amount of force without damaging the integrity (i.e. tearing) of either of the components. A normal amount of force is the amount of force a user would use to remove a component meant to be separated from another component without damaging either component. As used herein a “patient” includes a mammal such as a human being. All embolization devices as described herein are used for medical purposes and are therefore sterile. Although the drawings show certain elements of an embolization device in combination, it should be noted that such elements can be included in other embodiments or aspects illustrated in other drawings. In other words, each of the disclosed aspects and embodiments of the present disclosure may be considered individually or in combination with other aspects and embodiments of the disclosure

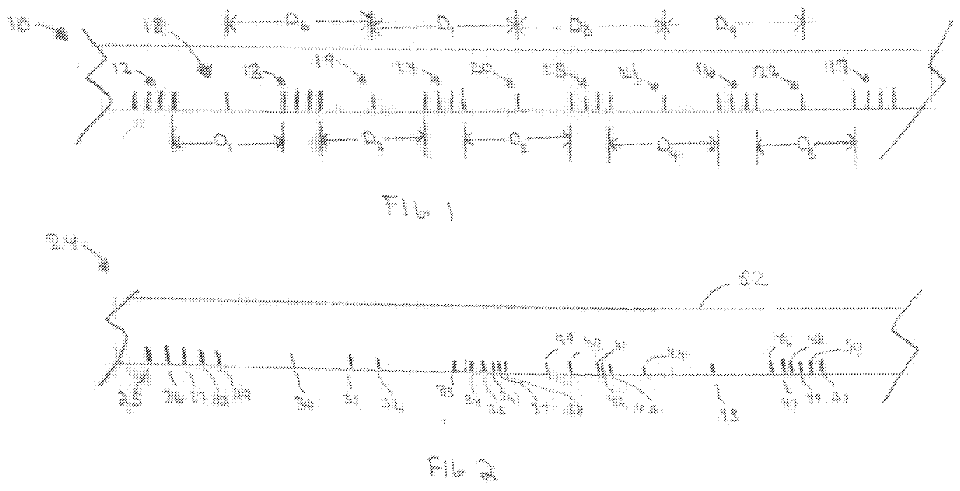

Embolization devices to occlude a bodily cavity are provided herein. The bodily cavity can be a lumen, such as vessel, or an outpouching, such as an aneurysm. The embolization devices can assume a highly complex higher-order structure and have a spectrum of topological configurations. In an aspect, the embolization device is a filament or hollow tube (referred to herein as a “hypotube”) that defines a plurality of variable notches. Notches include cuts, breaks, groove, channels, serrations, or other reductive surface alterations defined by the filament or hypotube. By “variable” is meant that the plurality of notches does not have or does not follow a uniformly repetitive pattern on the filament or hypotube but rather has a non-uniform pattern. FIG. 1 illustrates a hypothetical device 10 that has a uniformly repetitive pattern of notches 12-22. In particular, the distances D₁-D₅ between the sets of notches 12-17 is the same and the distances D₆-D₉ between individual notches 18-22 is the same. By way of contrast, FIG. 2 illustrates an embolization device 24 where the notches 25-51 are located on the filament or hypotube 52 and do not follow a uniform repetitive pattern. Although with respect to the entire length of an embolization device, some notches or set of notches may be equidistantly spaced apart, not all notches follow such an equidistant spacing pattern.

Further, the non-uniform pattern in which the notches are distributed on the filament or hypotube is not to limited to non-uniform spacing (e.g. non-uniform intervals or periodicity) of notches but can also include a non-uniform pattern of other parameters of the notches such as a non-uniform pattern of depths, shapes, orientations, and/or angles of the plurality of notches. Again, with respect to the entire length of an embolization device, some notches or set of notches may have similar depths, shapes, orientations, and/or angles, but not all the notches follow a uniform, repetitive pattern. Further, the non-uniform pattern of the plurality of notches at least partially contributes to the shape of the embolization device in a deployed state by selectively biasing the embolization device to assume a pre-defined shape. The pre-defined shape is ultimately selected based upon the configuration of the cavity in which the embolization device is deployed. As such, the plurality of notches provides a custom curvature at specific sections of the device. Such customization can allow for treatment of a particular aneurysm or vascular anomaly if the shape of the target deployment site is known beforehand, including custom generation of an embolic filament designed to conform to a unique cavity.

Mathematical modeling can be used to achieve complex, higher order geometric transformation of embolization devices provided herein. In particular, CAD/3D planning software can be used to determine the desired deployed shape of the embolization device and mathematical calculations can be performed to calculate the most appropriate shape, depth, spacing, and/or direction of the notches to achieve the desired deployed shape. As such, the embolization device can assume a predefined higher order shape with precise design features when placed in a constrained environment and/or when loading forces are applied to the embolization device.

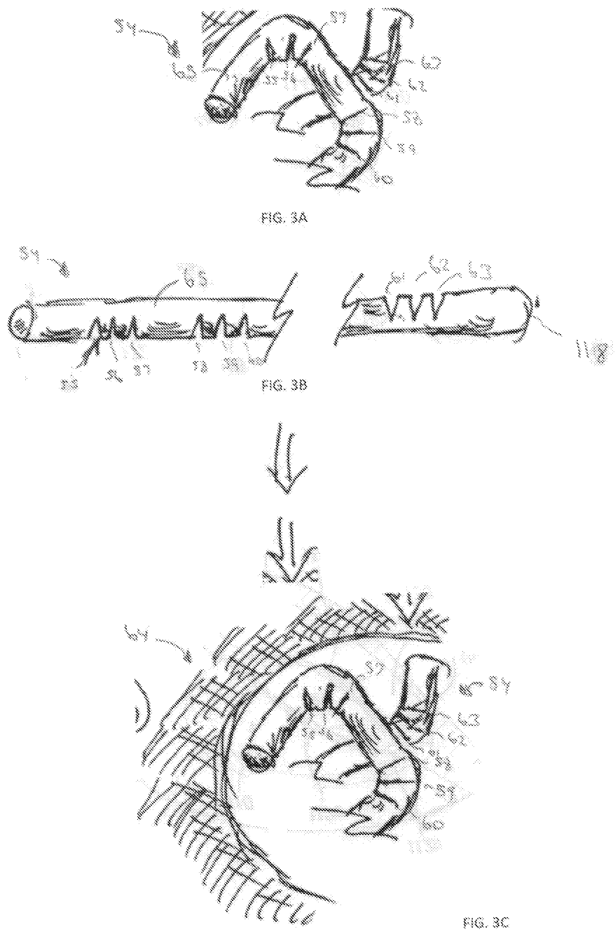

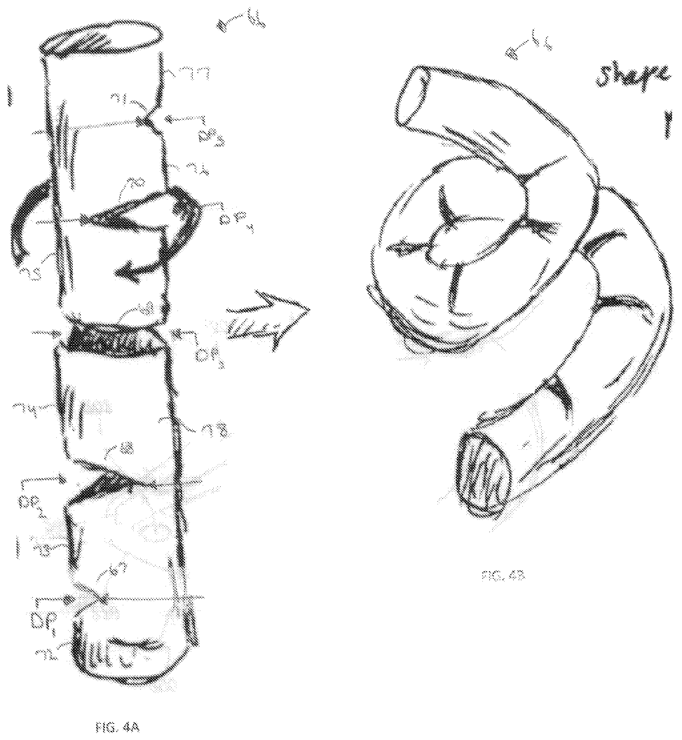

FIGS. 3A-3C depicts an embolization device 54 (or part thereof) comprising a filament or hypotube 65 having a plurality of notches 55-63 located at non-uniform variable intervals on the filament or hypotube 65. FIG. 3A depicts a pre-defined desired shape of embolization device 54. The pre-defined shape is based on the shape, size and other characteristics of the aneurysm 64 illustrated in FIG. 3C; FIG. 3B depicts embolization device 54 in a linear configuration, which is the configuration embolization device 54 assumes when it is loaded into a delivery device, such as, for example, a catheter, sheath or other tubular device; and FIG. 3C depicts embolization device 54 when deployed in an aneurysm 64. As can be seen from FIG. 3C, once deployed into an aneurysm, embolization device 54 takes on the pre-defined shape depicted in FIG. 3A. The variable nature of the notches facilitates conformal shaping of embolization device 54 when constrained within the space of aneurysm 64.

As stated above, the interval, depth, shape, face/orientation and/or angle of the notches can influence the manner in which the three-dimensional embolization device takes shape when deployed and can selectively bias the embolization device to assume a particular configuration along its length when subjected to stress forces or when placed in a constrained space. For example, the spacing of the notches can influence the constrained curvature of the embolization device when deployed. The orientation along a given surface, width, depth, and periodicity of the notches can deliberately bias or skew the curvature of the embolization device under a loading force.

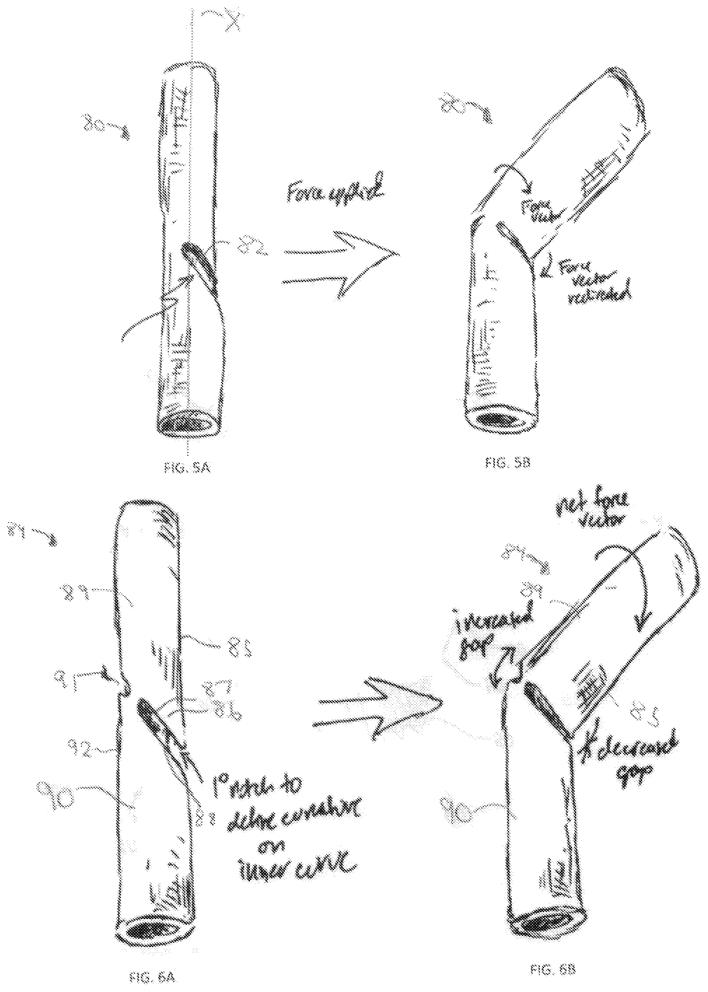

FIGS. 4A-4B illustrate an example of where embolization device 66 (or part thereof) has a plurality of notches 67-71 and at least some of the notches are situated on difference faces (DP₁-DP₅). FIG. 4A illustrates embolization device 66 when in a non-deployed linear shape and FIG. 4B illustrates embolization device 66 in a deployed state (e.g. when placed within a confined shape). When an axial load is applied, the embolization device can transition to a secondary structural embolization device 66, such as a helix, in an embodiment. The density, spacing, size of the notches, can determine the periodicity of the secondary structure, comprised in this instance of uniform segments 72-77.

Shallower notches, such as notches 67 and 71 provide more stiffness and less curvature of filament or hypotube 78 at the faces of adjacent sections 72 and 73 that define notch 67 and at the faces of adjacent sections 76 and 77 that define notch 71. Deeper notches, such as notches 68 and 69 provide more flexibility and greater curvature of filament or hypotube 78 at the faces of adjacent sections 73 and 74 that define notch 68 and at the faces of adjacent sections 74 and 75 that define notch 69. The depth of the notches can be set during manufacturing to control the degree of curvature of the filament or hypotube at certain sections of the filament or hypotube when the embolization device is deployed. The desired degree of curvature of certain sections of the embolization device can be determined based on the configuration of the aneurysm, or other bodily cavity, in which the embolization device is to be deployed to sufficiently occlude blood flow to the aneurysm, or other bodily cavity.

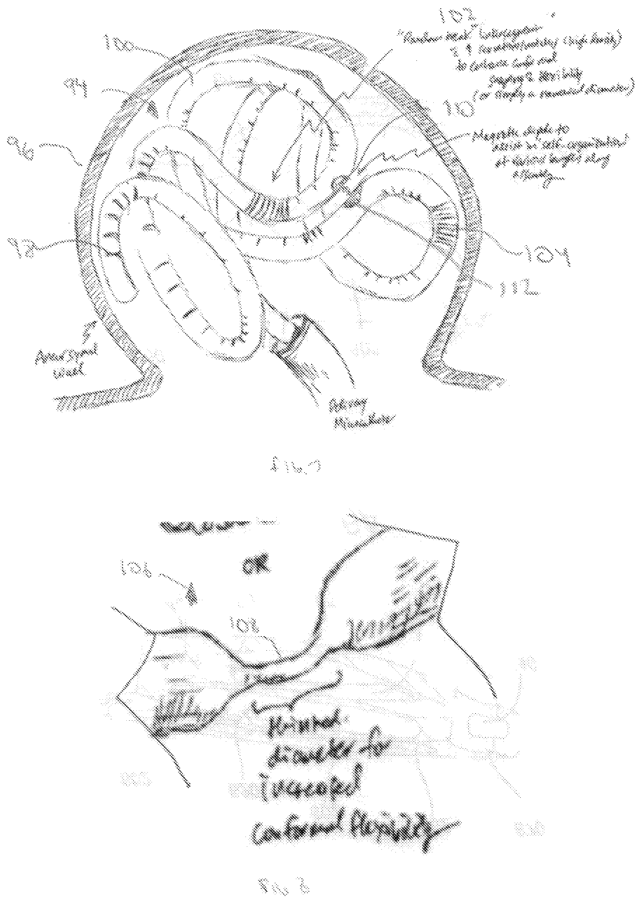

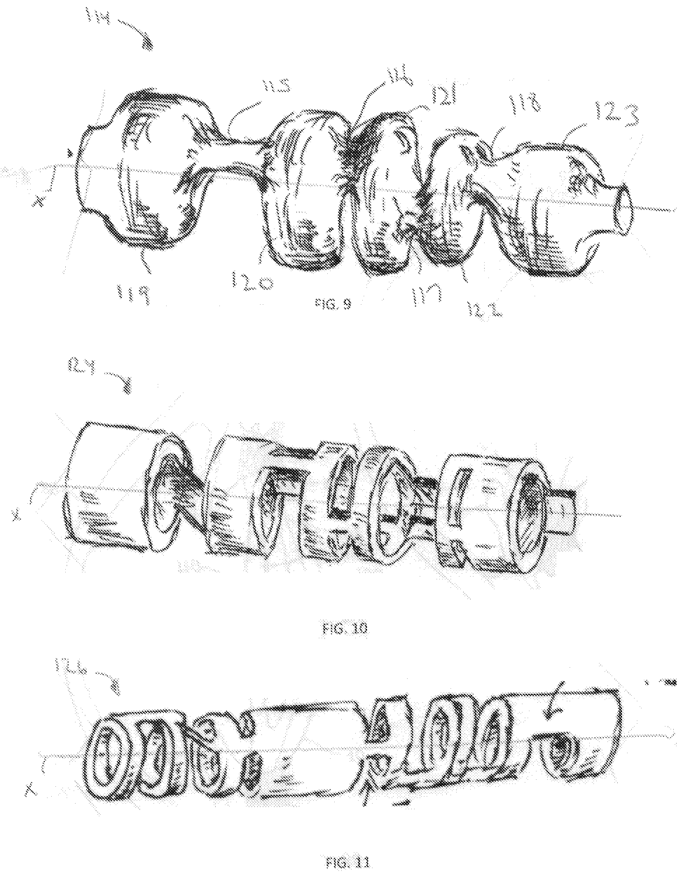

FIGS. 5 and 6 illustrate aspects of an embolization device where the angle of the notch can influence the bending or curvature of the filament or hypotube. FIGS. 5 illustrates a section of a hypotube 80 with only one notch for purposes of clarity but it is understood that the entire embolization device will have a plurality of notches. FIG. 5 depicts a notch 82 that has an oblique angle (with respect to the longitudinal axis X) of hypotube 80. FIG. 5A illustrates hypotube 80 in a non-deployed state and FIG. 5B illustrates hypotube 80 in a deployed or semi-deployed state. FIG. 6 depicts a section of a hypotube 84 that includes a primary notch 86 on a first side 85 of hypotube 84 to provide curvature or bending of hypotube 84 at the faces 87 and 88 of adjacent sections 89 and 90 that define notch 86 and a secondary “counter notch” 91 on a second side 92 of hypotube 84 to decrease material stress on the opposing face 92. Although FIGS. 5 and 6 illustrate a non-orthogonal configuration of the notch defined by a hypotube, such a notch can be defined by a solid filament as well.

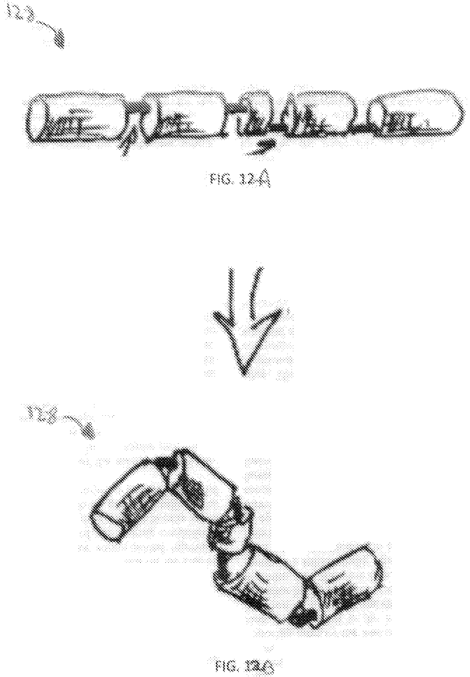

FIG. 7 illustrate an exemplary embolization device 94 having a complex fill shape deployed in an aneurysm 96. As can be seen, a plurality of notches 98 are non-uniform specifically distributed along filament or hypotube to achieve this structure 100. In this particular example, there are sections, such as sections 102 and 104, where there is an increased density of notches to enhance conformal shaping and flexibility of the hypotube at those sections. Alternatively, and with respect to FIG. 8, the diameter of a hypotube 106 can be smaller at certain sections, such as section 108, to enhance the conformal shaping and flexibility of the hypotube. In other words, the thickness of the embolization device can vary along its length facilitating some non-uniformization or increased flexibility in certain areas to conform to a given target volume.

Regarding thickness, an embolization device can include portions of reduced thickness and such portions can be located in different locations of the embolization device and can have different lengths. For example, sections can be of smaller diameter than adjacent sections or be offset or eccentric relative to the longitudinal axis of the device in order to bias the embolization device and promote curvature in transition zones from one section to another.

FIG. 9 illustrates an embolization device (or part thereof) 114 that has portions of reduced diameters such as portions 115-118 compared to the diameters of portions 119-123. Further, at least some portions 115-118 do not extend in the same plane and are offset from one another with respect to longitudinal axis X. For example, portion 115 is offset from portion 116, which is offset from portion 116, which is offset from portion 117, which is offset from portion 118. Further, the lengths of portions 115-118 are not necessarily the same as seen in FIG. 9. FIGS. 10 and 11 illustrate other exemplary embolization devices (or parts thereof) 124 and 126 respectively where the device has areas of reduced thickness relative to adjacent portions of the embolization device as well as varying angles relative to the longitudinal axis X and varying lengths and locations. All such parameters (e.g. length, angle, location) can be modulated to selectively bias the curvature of the embolization device at such areas of reduced thickness. For example, FIGS. 12A-12B illustrate an embolization device 128 having portions of varying thicknesses and located at varying locations. FIG. 12A illustrates embolization device 128 in a non-deployed linear configuration and FIG. 12B illustrated embolization device 128 in a deployed configuration, such as when confined within a constrained space. The above embolizations devices are only exemplary and other embolization devices can have other thickness patterns.

The density, shape, depth and/or periodicity of the notches combined with material from which the embolization device is fabricated can influence the softness/stiffness/handling characteristics of the deployed embolization device. Depending on the shape, depth, size, and/or interval of the notches and the primary material characteristics, the composition of which can be varied along the length of the device, the bending of the device may occur on the inner curve vs. the outer curve of the notches.

By varying the shape, periodicity and other above-mentioned parameters of the notches, the embolization device can have variable stiffness/softness characteristics along the length of a filament or hypotube even while maintaining identical filament or hypotube diameter and composition. Again, this does not preclude varying the material composition along its length. This allows for pre-defined regions of structure (“frame”) and softness (“fill”) that have non-uniform shapes. All these characteristics can be achieved despite employing the same filament or hypotube. The embolization device can be a combination of a “frame” section and a “filling” or “finishing” section. For example, the embolization device can serve as a frame at one end that is highly organized and stiffer distally with a filling or finishing section at the other end that is very soft, has a helical shape, and also serves as a variable detachment zone. For example, a very soft “coil” behavior can be achieved at the terminal end with closely spaced notches without even modifying the composition of the filament or hypotube or its diameter. Alternatively, the embolization device can have a wider diameter distal end for increased stiffness for a “frame” and then can transition to a narrower diameter for finishing properties.

An embolization device can have “cassettes” or “structural modules that are self-organizing elements (SOE) or structural domains that are embedded as repeating or varying units along the longitudinal axis of the device. Such cassettes can achieve higher-order structures spontaneously when placed in a partially/semi-constrained environment/surroundings. The cassettes can be designed in various ways For example, a 7 mm loop module can be connected to a 6 mm module oriented at a 90 degree angle, then a 5 mm module, etc. In doing so, the embolization device has an element of ‘non-non-uniformness’ due to the deliberate, pre-specified placement of notching dispersed along the longitudinal axis of the filament or hypotube in order to achieve a predetermined conformation (within an envelope of degrees of freedom) when placed in a constrained environment. Not just the sequence of notches, but their precise, respective positions on the face of the filament in 360 degrees helps achieve this, as illustrated in 4A.

In certain aspects, the embolization device can include magnets to facilitate self-organization of the embolization device. For example, referring to FIG. 7, magnetic dipoles 110 and 112 can be embedded in the filament or hypotube at specific intervals to encourage ‘self-assembly’ into a higher order structure with a lower energy state. In addition or alternatively, the embolization device may include textural or structural features that can facilitate self-adherence at periodic intervals.

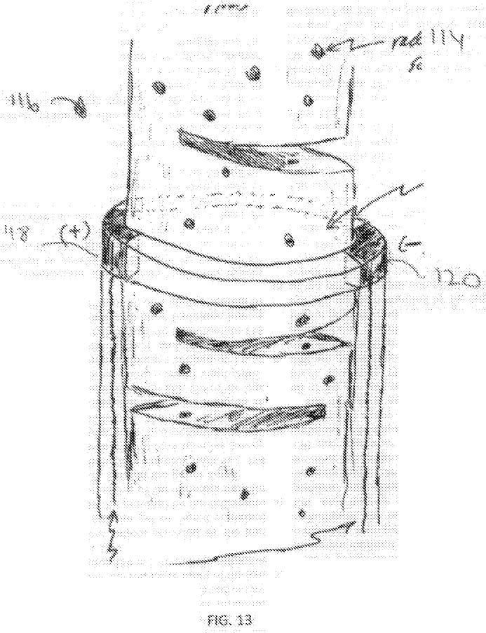

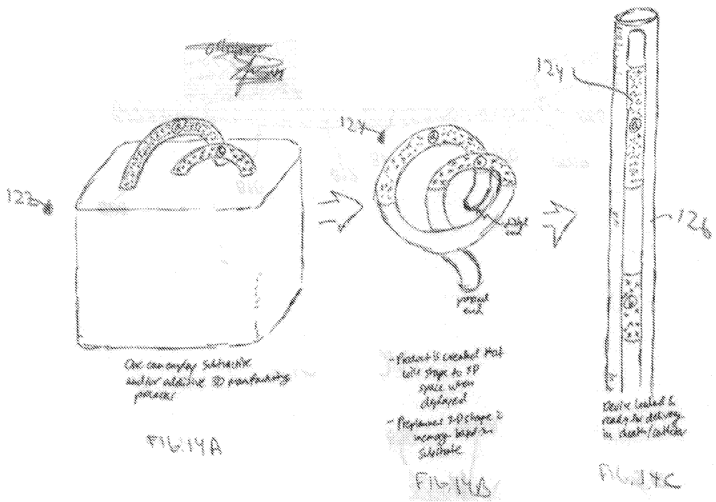

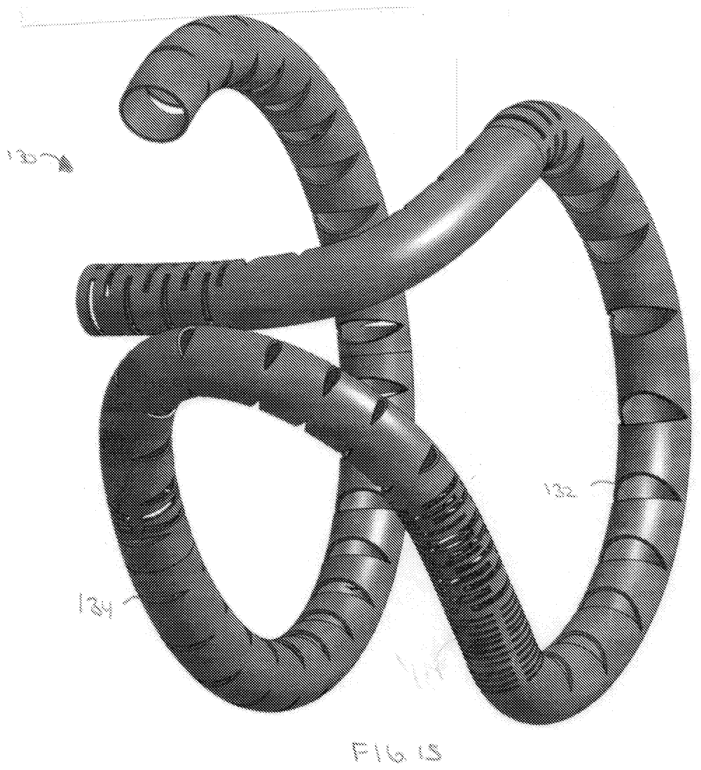

An embolization device as disclosed herein can be fabricated, for example, from biocompatible polymers; metals; metal alloys including platinum iridium, platinum tungsten or nickel titanium; and suitable combinations thereof. All portions of the embolization device need not be fabricated from the same material. In certain aspects, the distal end of the embolization can be fabricated from a stiff, resistant polymer and/or metal and the middle portion and the proximal end can have different characteristics as well. The embolization device can be biodegradable or resorbable; fabricated from a polymer mix including radiodense/radioactive materials such as a polymer with tungsten/tantalum powder embedded in the mixture to improve visualization; fabricated from a polymer that can be cured with ultraviolet (UV) light in its final configuration; and/or fabricated from fluorescent or luminescent material. For example, referring to FIG. 13, radiodense markers 114 can be embedded in the filament or hypotube 116 to enhance radiographic/fluoroscopic visualization.

In certain aspects, the embolization device is fabricated from a shape memory material or have portions fabricated from a shape memory material. As such, the embolization device can retain conformational memory of a complex space-filling shape. In other words, the embolization device can be shaped into a complex space-filling shape and following deployment and removal from a linear-constrained space, such as a catheter or sheath, the embolization device can re-assume its original shape based on the memory properties of the embolization device. For example, the distal, mid, and/or proximal ends of the embolization device can have predefined shape memory curves such that they hold a shape spontaneously once out of the delivery catheter. For example, the distal end can have a predefined shape memory curve such that the initial distal end does not have to contact the aneurysm wall in order to achieve the desired shape and can minimize the risk of dome puncture. Similarly, the proximal end (at the detachment zone for example) can have a pre-defined memory shape such that once detached, the device will not straighten. This can minimize a “tail” formation of the shaped embolization device. In another instance, the distal end memory shape of the embolization device can assist in positioning the microcatheter itself into the aneurysm.

The embolization device can include other characteristics, features and components. For example, the embolization device can include bioactive substances such as, for example, peptides, pro-thrombotic (thrombogenic) agents, anti-thrombotic agents, biologically active proteins/DNA/RNA, and suitable combinations thereof. The embolization device or parts thereof may have a hydrophilic coating, which expands on contact with the blood, and can allow the device to achieve spontaneous pre-determined three dimensional complex shapes based upon the positioning of notches or serrations in the linear structure of either the coating or the primary underlying filament.

The filament or hypotube can have a fixed or variable detachment zone (such as detachment zone 118 referenced in FIG. 3B). The filament or hypotube can be sensitive or conductive to heat or light to allow for selective detachment of the embolization device. For example, the filament can include a detachment zone that allows for thermal detachment or a dissociation mechanism (electrolytic) based on conductivity, for example. This would allow for a custom detachment system and can allow for detachment of a variable length filament (such length can be the length at which the aneurysm is obliterated based on radiographic or other imaging). For example, referring to FIG. 13, the filament or hypotube 116 can comprise a conductive substrate and the distal end of the device delivery catheter can include embedded anodes 118 and cathodes 120, allowing selective detachment at that point. The embolization device can be delivered, for example, through a device positioning system (DPU) or a microcatheter including a custom delivery microcatheter. For example, the terminal end of a microcatheter can have a thermal or electrolytic heater to allow melting of the embolization device at a detachment zone to provide detachment at a given/desired length of the filament or hypotube.

The embolization device itself may be able to guide itself into the aneurysm dome if a microcatheter brings it into proximity, as the filament or hypotube's shape is conferred by extrinsic pressure from the aneurysm wall. Alternatively, there may be shape-memory in parts of the embolization device to assist in guiding the microcatheter into the target lumen by virtue of the leading terminus of the device, e.g. the embolization device can guide the microcatheter close to the aneurysm, and the embolization device can be able to reach into the aneurysm, where it can be deployed.

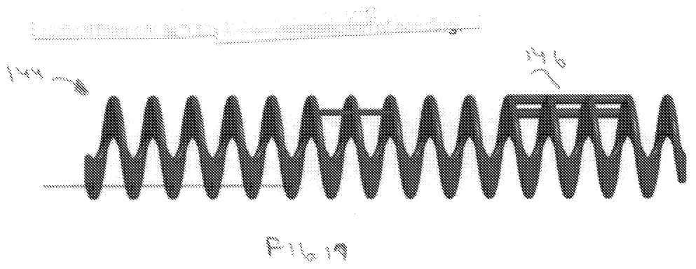

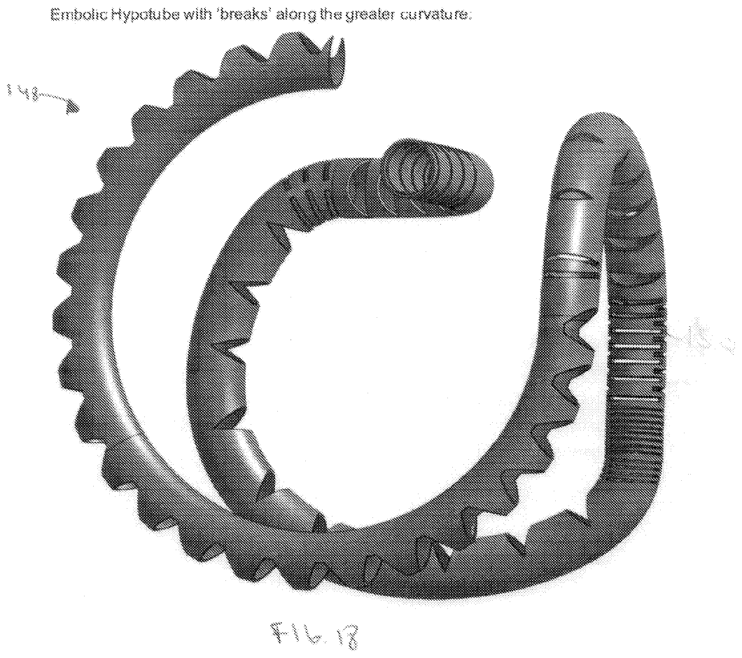

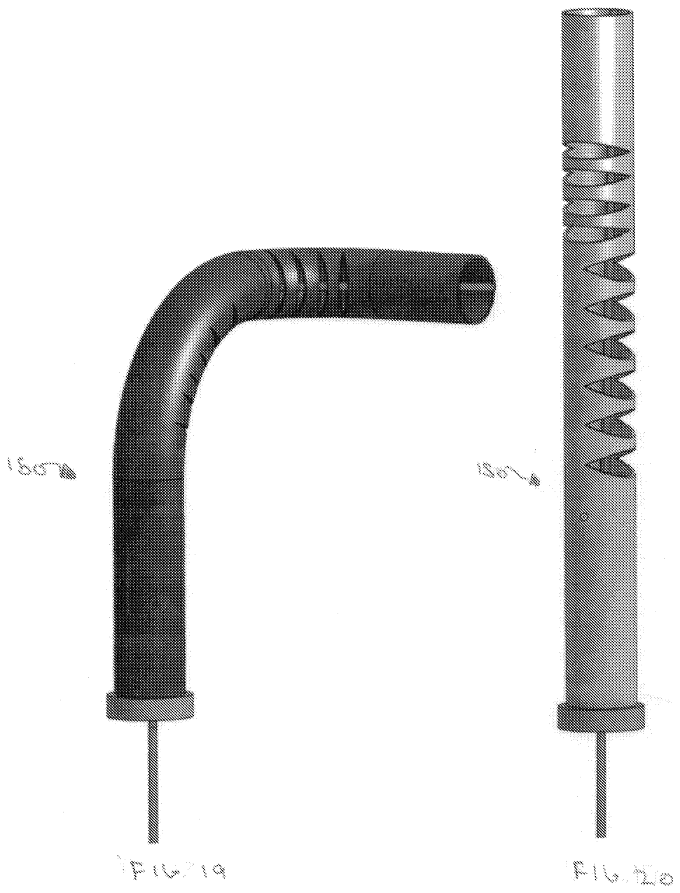

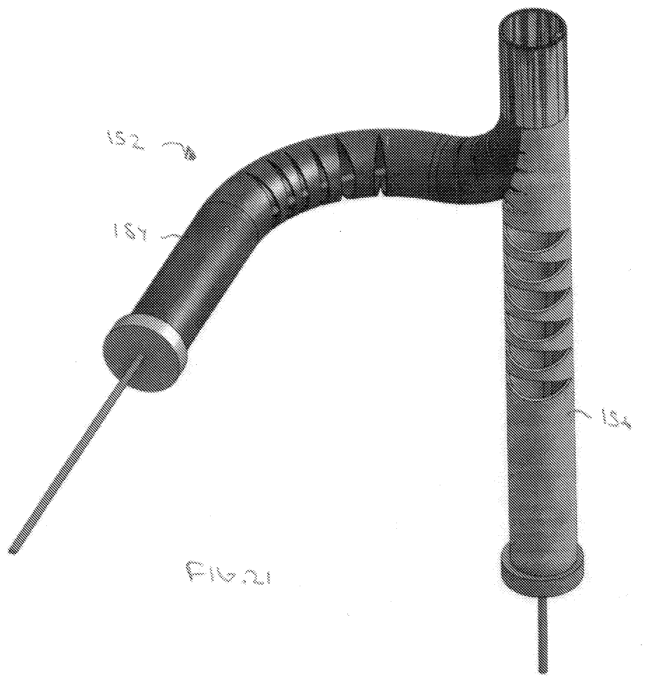

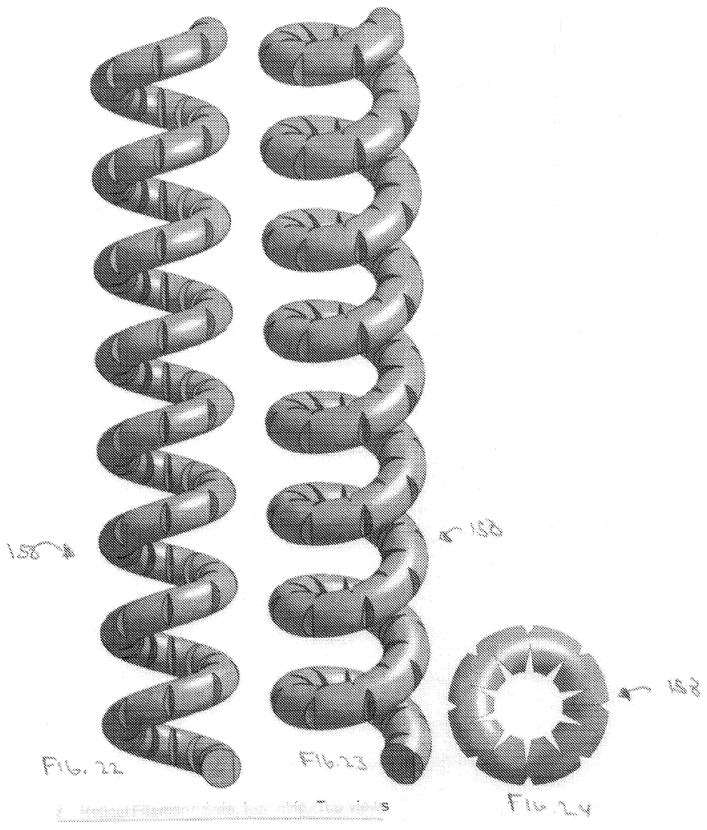

FIGS. 15-22 illustrate different embodiments of an embolization device. FIG. 15 is an example of an embolization device 130 with deeper notches 132 along the less curvature and shallower “tension releasing” notches along the greater curvature to pre-define a curve. FIG. 16 is an example of an embolization device 136 with flexible linker 138, selective notching defining greater and lesser curvature diameters 140, and accordion-type transition elements 142. FIG. 17 is an example of an embolization device 144 in the form of a helical filament with struts that bias the direction of bending. FIG. 18 is an example of an embolization device 148 with breaks along the greater curvature. FIG. 19 is an example of a steerable hypofilament with tension applied and FIG. 20 is an illustration of the same hypofilament in a resting state (no tension applied). FIG. 21 is an example of an embolization device 152 that is steerable hypofilament with the left section 154 illustrating the device when tension is applied and the right section 156 in a resting state (no tension applied). FIGS. 22 and 23 are opposing side views of an exemplary embolization device in the form a helical filament and FIG. 24 is a top view of the same embolization device.

The endovascular device can be manufactured by extrusion, 3D printing techniques, die molding, injection molding or other suitable manufacturing methods. The configuration of the embolization device when in a deployed state can be customized and pre-planned for a particular type of aneurysm or vascular anomaly. The notches can be created by methods such as lithography including laser lithography, etching, photo-resistive substrate manufacturing, and the like. The flexibility of the device can be achieved through subtractive or selective removal or the substrate or strictly via additive synthesis. The notches can be created during or after extrusion is completed, for example. The non-uniform distribution of the plurality of notches can be predefined at the time of manufacturing, such as during extrusion or post-formation of a polymer or metal column substrate. The notches can be created by rotating the extruded substrate and generating notches at specific intervals and controlling the face of the serrating or by performing controlled notching along the length of the substrate after it has been formed. For example, a cutting device can be used to make cuts in the substrate at fixed angles or the substrate can be rotated and cut at specific positions along the substrate's axis. “Counter” notches (as described above) can be etched, carved, cut, etc. in order to relieve stress on the outer curvature of the bend of the embolization device. Segments can also be cut on a notching die to manipulate larger spans of the substrate.

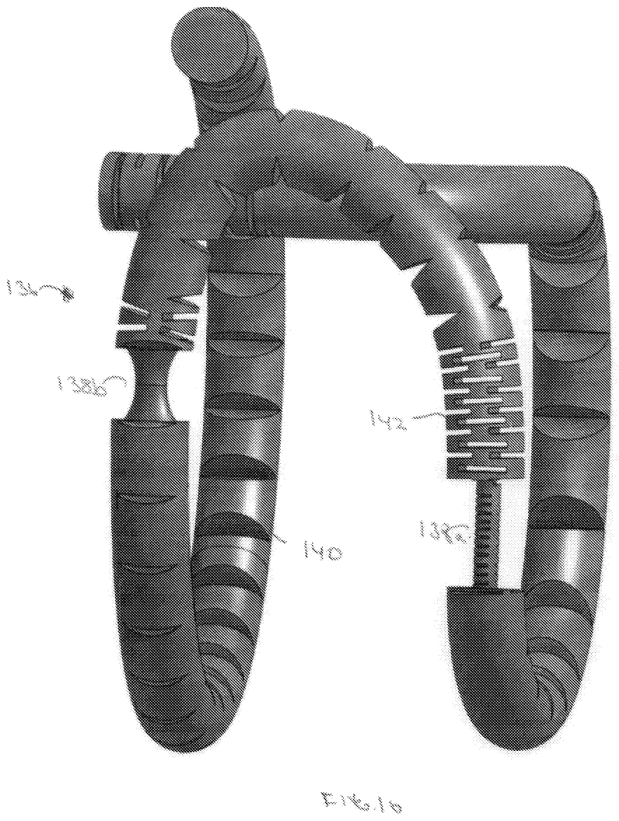

The embolization device can be formed or heat-treated in a mold filament to confer a memory to the device and can obviate the need for manual complex mandrel winding. This can help bias the embolization device into a pre-defined shape even without being constrained in a specific space. For example, FIG. 14A illustrates such a mold with subtractive or additive surfaces to manufacture an embolization device 124. Such embolization device 124 with a non-uniform pattern of notches, when removed from the mold as illustrated in FIG. 14B, will have a shape that will conformally shape to the three-dimensional space in which the device is deployed. FIG. 14C illustrates embolization device 124 loaded into a delivery device 126, such as a sheath or catheter. As can be seen from this figure, embolization device has a substantially linear configuration when loaded into a delivery device. The embolization device can have a circular or polygonal cross-sectional shape such as triangular cross-sectional shape or other suitable shapes. The filament or hypotube can be rotated during extrusion to create a spiral shape similar to DNA. In embodiments where the embolization device is a filament, the filament can be a monofilament or a polyfilament, the latter of which can increase the strength and stretch-resistance of the embolization device. In embodiments where the embolization device is a hypotube, such a configuration can reduce the overall mass of the device and can facilitate delivery of a fluid through the device during deployment to reduce resistance.

The embolization device, once formed in its linear shape, can be loaded into a “shaping volume” (e.g. a cavity mold to assist in guiding formation of the shape when an axial load is applied), followed by heat/cold/steam/light-treated or other energy treatment. Shape-memory is then retained for deployment into a targeted cavity within the body. This allows shape-memory preservation without the need for a mandrel, as well as either avoidance or minimization of axial-pressure loading in vivo in order to achieve the targeted conformation. This approach can increase throughput for manufacturing of the embolization device by reducing the complexity of shape creation whilst reducing axial loading requirements upon deployment. This is particularly advantageous to reduce wall shear stress in a ruptured or unruptured aneurysm treatment, for example.

The tertiary conformation (3D) structure achieved by the embolic filament or hypotube can be influenced by the shape of the volume to be filled; the loading force subjected to the filament; the intrinsic stiffness of the filament itself; the shape memory of the filament itself; the specific orientation along a given face, the shape, density, and depth of the notching along the length of the filament; or combinations thereof.

The embolic filament or hypotube can ultimately achieve the lowest energy configuration for a given space. This is what can ultimately govern the final shape of the device, once placed into an aneurysm. The precise spatial arrangement of the notches along the length of the filament can dictate the bias towards a particular higher order structure. Given that there can no longer be constraint by manual mandrel winding, as is done in the creation of conventional coils, higher order structures can be achieved which can preferentially fill an ellipsoid shape or a spherical shape, for example, and pre-determined complex 3D structures otherwise previously difficult or impossible to achieve via manual assembly of coils.

Once the underlying material handling characteristics of a given material with specific notch density, depth, spacing, and orientation are known, modular “structural cassettes” can be engineered so that the filament can be biased to achieve a specific higher order shape when deployed. For example, a structural cassette can be created that preferentially assumes a 9-10 mm loop horizontally at its distal most end, followed by a cassette with notches oriented 90 degrees to that original module that generate a 6-8 mm loop more proximally, and so forth. There may be ‘transition zones’ interposed between these repeating elements (functional cassettes) such as ‘random break’ spacers, flexible linkers, or spans in which the orientation of the notches is angled or spaced by varying degrees along the faces to create helices or gradual turns in three-dimensional space. A fish-mouthed notch can create a segment with torsional rotation about that point, redirecting the filament laterally locally based upon the net forces locally encountered. As does when an oblique notch generates a turn in the filament, akin to changing the force vector.

In one embodiment, an embolic filament can be engineered which performs an ‘outside-in’ filling of a 3D space, in which the most distal loops are the largest, followed by successively smaller proximal loops which nest within the superstructure to obliterate an aneurysm or cavity. The material handling characteristics can be modulated such that the filament is firmer distally, and softer more proximally, so that the terminus of the coil, closest to the detachment zone places the least stress upon the aneurysm walls.

Although the above embolization device has been described mainly with respect to obliterating an aneurysm, the device can be used to occlude space in other cavities within the body. For example, the embolization device can be used for treating other vascular malformation or anomalies. Further, a larger scaled version of this device can enable enhanced flexibility of a wire or catheter for endovascular treatment, the latter embodiment may incorporate coverage of a hypotube scaffold with a polymer sheath. The advantage again is that such a design can confer flexibility without the risk of stretching or unwinding.

Each of the disclosed aspects and embodiments of the present disclosure may be considered individually or in combination with other aspects and embodiments. Further, while certain features of embodiments may be shown in only certain figures, such features can be incorporated into or deleted from other embodiments shown in other figures or otherwise disclosed in the specification. Additionally, when describing a range, all points within that range are included in this disclosure.

What is claimed is:

1. An embolization device to occlude a cavity within the body comprising:
a filament or hypotube having a longitudinal axis extending therein;
a plurality of notches distributed in non-uniform pattern on the filament or hypotube, the non-uniform pattern at least partially contributing to the shape of the embolization device in a deployed state by selectively biasing the embolization device to assume a pre-defined shape, the pre-defined shape being based on the configuration of the cavity in which the embolization device is deployed.

2. The embolization device of claim 1, wherein the non-uniform pattern comprises a non-uniform pattern of spacing, angle, orientation, depth, and/or shape of the plurality of notches.

3. The embolization device of claim 1, wherein at least some of the plurality of notches extends in a direction different than the direction in which the longitudinal axis extends.

4. The embolization device of claim 1, wherein the cavity within the body is an aneurysm and the pre-defined shape is based on the configuration of the aneurysm in which the embolization device is deployed.

5. The embolization device of claim 1, wherein the filament or hypotube comprises a plurality of segments having varying diameters.

6. The embolization device of claim 5, wherein the filament or hypotube has a non-uniform pattern of the plurality of segments having varying diameters.

7. The embolization device of claim 6, wherein some of the plurality of segments are offset or eccentric relative to the longitudinal axis of the filament or hypotube.

8. The embolization device of claim 1, wherein the filament or hypotube comprises a shape memory material.

9. The embolization device of claim 1, wherein the filament or hypotube comprises magnetic dipoles on opposing sides thereof to facilitate self-organization of the embolization device in the cavity within the body.

10. The embolization device of claim 1, wherein the cavity within the body is an aneurysm and the pre-defined shape is a complex fill shape.
